(19) United States
(12) Patent Application Publication
(10) Pub. No.: US 2006/0194810 A1
(43) Pub. Date: Aug. 31, 2006

(54) Methods of treating ischemic related conditions

(76) Inventors: Bijan Almassian, Cheshire, CT (US); Zhi-Gang Jiang, Gaithersburg, MD (US); Michael S. Lebowitz, Baltimore, MD (US); Weiying Pan, Cockeysville, MD (US); Hossein A. Ghanbari, Potomac, MD (US)

Correspondence Address: M. Elisa Lane, 16520 Montecrest Lane, Darnestown, MD, 20878, US

(21) Appl. No.: 11/282,314

(22) Filed: Nov. 18, 2005

Related U.S. Application Data
(63) Continuation-in-part of application No. 10/835,668, filed on Apr. 30, 2004.

(51) Int. Cl. A61K 31/4965; A61K 31/44; A61K 31/426; A61K 31/4172

(52) U.S. Cl. 514/252.1; 514/357; 514/365; 514/400

(57) ABSTRACT

The present invention relates to methods of treating or preventing ischemia-related (i.e., neural cell hypoxia and/or hypoglycemic) conditions by administering to a patient in need thereof certain thiosemicarbazone compounds. More particularly, the present invention relates to methods of preventing or treating certain ischemia-related conditions, which may include Alzheimer's disease, Parkinson's disease, and ischemic states that are due to or result from such conditions as: coronary artery bypass graft surgery; global cerebral ischemia due to cardiac arrest; focal cerebral infarction; cerebral hemorrhage; hemorrhage infarction; hypertensive hemorrhage; hemorrhage due to rupture of intracranial vascular abnormalities; subarachnoid hemorrhage due to rupture of intracranial arterial aneurysms; hypertensive encephalopathy; carotid stenosis or occlusion leading to cerebral ischemia; cardiogenic thromboembolism; spinal stroke and spinal cord injury; diseases of cerebral blood vessels, e.g., atherosclerosis, vasculitis; macular degeneration; myocardial infarction; cardiac ischemia; and superaventicular tachyarrhytmia.

### FIELD OF THE INVENTION

The present invention relates to methods of treating ischemia-related diseases and disorders, including neuronal and cardiac diseases due to sudden loss of oxygen, as well as degenerative diseases, such as, Alzheimer's disease. The methods involve the use of certain thiosemicarbazone compounds.

### BACKGROUND OF THE INVENTION

The present invention is broadly directed to a new use of certain N-heterocyclic carboxaldehyde thiosemicarbazones (HCTs), which have up to now been known as useful as antineoplastic agents, acting as potent inhibitors of ribonucleotide reductase. Methods of treatment of tumors using such compounds are disclosed inter alia in U.S. Pat. Nos. 5,721,259 and 5,281,715 of Sartorelli et al. Further, the present invention is directed to a number of new analogues of the HCTs, which surprisingly have been found as neuroprotective.

More recently, U.S. Pat. No. 6,613,803 disclosed the use of certain novel thiosemicarbazones for the treatment of neuronal damage and neurodegenerative diseases. The novel compounds are described as exerting their therapeutic effects as sodium channel blockers.

However, until now there has been no disclosure in the art of the use of compounds that are the same or similar to those disclosed in the Sartorelli patents for treating or preventing neuronal damage.

Nerve cells require energy to survive and perform their physiological functions, and it is generally recognized that the only source of energy for CNS neurons is the glucose and oxygen delivered by the blood. If the blood supply to nerve tissue is cut off, neurons are deprived of both oxygen and glucose (a condition known as ischemia, and which is used herein synonymously with deprivation of oxygen and/or glucose), and they rapidly degenerate and die. This condition of inadequate blood flow is commonly known in clinical neurology as “ischemia.” If only the oxygen supply to the brain is interrupted, for example in asphyxia, suffocation or drowning, the condition is referred to as “hypoxia”. If only the glucose supply is disrupted, for example when a diabetic takes too much insulin, the condition is called “hypoglycemia”.

In recent years, it has been learned that glutamate, which functions under normal and healthy conditions as an important excitatory neurotransmitter in the central nervous system, can exert neurotoxic properties referred to as “excitotoxicity” if ischemic conditions arise. Normally, glutamate is confined intracellularly, and is only released from nerve cells at a synaptic junction in tiny amounts for purposes of contacting a glutamate receptor on an adjacent neuron; this transmits a nerve signal to the receptor-bearing cell. Under healthy conditions, the glutamate released into the extracellular fluid at a synaptic junction is transported back inside a neuron within a few milliseconds, by a highly efficient transport process.

The excitotoxic potential of glutamate is held in check as long as the transport process is functioning properly. However, this transport process is energy dependent, so under ischemic conditions (energy deficiency), glutamate transport becomes inadequate, and glutamate molecules released for transmitter purposes accumulate in the extracellular synaptic fluid. This brings glutamate continually in contact with its excitatory receptors, causing these receptors to be excessively stimulated, a situation that can literally cause neurons to be excited to death. Two additional factors complicate and make matters worse: (1) overstimulated neurons begin to release excessive quantities of glutamate at additional synaptic junctions; this causes even more neurons to become overstimulated, drawing them into a neurotoxic cascade that reaches beyond the initial zone of ischemia; and, (2) overstimulated neurons begin utilizing any available supplies of glucose or oxygen even faster than normal, which leads to accelerated depletion of these limited energy resources and further impairment of the glutamate transport process.

Thus, energy deficiency conditions such as stroke, cardiac arrest, asphyxia, hypoxia or hypoglycemia cause brain damage by a two-fold mechanism; the initial causative mechanism is the ischemia itself, which leads to failure of the glutamate transport system and a cascade of glutamate-mediated excitotoxic events that are largely responsible for ensuing brain damage.

In addition to the conditions already mentioned, it has recently become recognized that various defects in the neuron's ability to utilize energy substrates (glucose and oxygen) to maintain its energy levels can also trigger an excitotoxic process leading to death of neurons. It has been postulated that this is the mechanism by which neuronal degeneration occurs in such neurological diseases as Alzheimer's, Parkinson's, Huntington's and amyotrophic lateral sclerosis (ALS).

For example, evidence for defective intracellular energy metabolism has been found in samples of tissue removed by biopsy from the brains of patients with Alzheimer's disease and this has been proposed as the causative mechanism that triggers an unleashing of the excitotoxic potential of glutamate, with death of neurons in Alzheimer's disease thereby being explained by an energy-linked excitotoxic process. Evidence for an intrinsic defect in intracellular energy metabolism has also been reported in Parkinson's disease and Huntington's chorea.

While no therapy for neuroprotection is currently marketed, there are drugs approved for use in the therapy of chronic neurological conditions, which are glutamate receptor (NMDA) antagonists. Although there is evidence of ameliorating affects of such drugs in chronic CNS degenerative states, it does not appear that NMDA antagonists, alone, can provide substantial protection against ischemia, generally, especially in an acute situation.

A significant limitation of glutamate receptor antagonists as neuroprotectants against ischemic neurodegeneration is that they appear to insulate the neuron against degeneration only temporarily; they do not do anything to correct the energy deficit, or to correct other derangements that occur secondary to the energy deficit. Therefore, although these agents do provide some level of protection against ischemic neurodegeneration, the protection is only partial and in some cases may only be a delay in the time of onset of degeneration.

Since neurons begin to degenerate very rapidly after the onset of an ischemic state, there is clearly a need for therapeutic agents that will actively protect neurons from further degeneration and death by, for example, restoring the energy balance provided by oxygen and glucose in the bloodstream. Such therapeutic agents could not only be used for acute instances of ischemia, but also preventing neuronal degeneration in chronic degenerative disorders, such as Alzheimer's and Parkinson's diseases on the basis of correcting neuronal energy deficiency and prevention of excitotoxic neuronal degeneration.

Further, the compounds of the present invention can also be used to treat neurological disorders of the ear and eye that result from ischemic-like etiology, as well as diabetic neuropathies.

The development of therapeutic agents capable of preventing or treating the consequences of ischemic events, whether acute or chronic, is highly desirable.

### SUMMARY OF THE INVENTION

The present invention relates to methods of preventing and/or treating disorders resulting from ischemic conditions by administering to a patient in need of such treatment certain N-heterocyclic 2-carboxaldehyde thiosemicarbazones (HCTs) and pharmaceutically acceptable salts or prodrugs thereof: Such useful compounds are encompassed by Formula I:

More preferably, the compound is selected from a compound of Formula II, below:

More preferably, the methods of the present invention employ a compound selected from:

(Specific Ones Used)

As a most preferred embodiment, PAN-811 (3-aminopyridine-2-carboxaldehyde thiosemicarbazone) is used to practice the methods of the present invention, which has the formula:

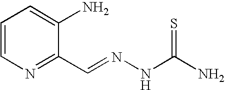

The present invention is also directed to methods of treating, ameliorating, and/or preventing specific ischemia-related conditions, including but not limited to treatment of neuronal damage following global and focal ischemia from any cause (and prevention of further ischemic damage), treatment or prevention of otoneurotoxicity and of eye diseases involving ischemic conditions (such as macular degeneration), prevention of ischemia due to trauma or coronary bypass surgery, treatment or prevention of neurodegenerative conditions such as amyotrophic lateral sclerosis (ALS), Alzheimer's disease, Parkinson's disease, and Huntington's chorea, and treatment or prevention of diabetic neuropathies.

### BRIEF DESCRIPTION OF THE DRAWINGS

FIG. 1 contains graphic representations of cell viability (left panel) and neuroprotective capacity (right panel) after pre-treatment with PAN-811 (A) or known neuroprotectants Vitamin E (B), lipoic acid (C), or Ginkgo biloba (D) and subsequent treatment with H₂O₂.

FIG. 2 contains graphic representations of the effects of PAN-811 on ROS generation in neuronal cells. (A); the effects of PAN-811 on H₂O₂-induced ROS generation in neuronal cells. (B); the effects of PAN-811 on the basal level of ROS generation in neuronal cells.

FIG. 3 is a graphic representation of the dependence of neurotoxicity on the concentration of glucose in hypoxic conditions.

FIG. 4 shows representative histological photographs of cells under hypoxic conditions with and without neuroprotectants, MK801 and PAN-811.

FIG. 5 is a graphic representation of the neuroprotective effects of PAN-811 under normoxic and hypoxic conditions.

FIG. 6 depicts graphic representations of the toxicity of PAN-811, under hypoxic/hypoglycemic conditions.

FIG. 7 is a graphic representation of the protective effects of PAN-811 on neuronal cell death due to mild hypoxic/hypoglycemic conditions.

FIG. 8 is a graphic representation of the neurotoxicity of PAN-811 where cortical neurons were treated with PAN-811 for 24 hours.

FIG. 9 is a graphic representation of the protective effects of PAN-811 against toxicity due to ischemia.

FIG. 10 shows graphic representations of cell viability after pre-treatment with PAN-811 or solvent and treatment with H₂O₂.

FIG. 11 shows graphic representations of cell viability after pre-treatment with PAN-811 or solvent and treatment with H₂O₂.

### DETAILED DESCRIPTION OF THE INVENTION

Ischemia-related disorder/disease pathologies involve a decrease in the blood supply to a bodily organ, tissue or body part generally caused by constriction or obstruction of the blood vessels as, for example, retinopathy, acute renal failure, myocardial infarction and stroke. They can be the result of an acute event (e.g., heart attack or stroke) or a chronic progression of events (e.g., Alzheimer's or ALS). The present invention is intended to be applicable to either acute or chronic pathologies.

The present invention relates to methods of treating ischemia-related conditions, particularly to neuronal cells and tissue, by administering to a patient in need of such treatment a compound of Formula I, or pharmaceutically acceptable salts or prodrugs thereof: 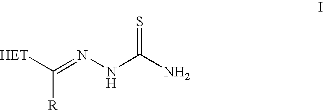
where HET is a 5 or 6 membered heteroaryl residue having 1 or 2 heteroatoms selected from N and S, and optionally substituted with an amino group; and R is H or C₁-C₄-alkyl.

In one preferred embodiment, the compound is of Formula II: 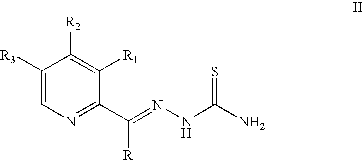
where R is H or C₁-C₄-alkyl; and R₁, R₂ and R₃ are independently selected from H and amino.

In another preferred embodiment, the compound is of Formula III: 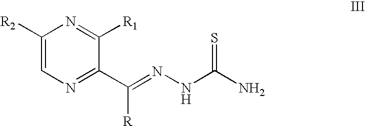
where R is H or C₁-C₄-alkyl; and R₁ and R₂ are independently selected from H and amino.

In another preferred embodiment, the compound is of Formula IV: 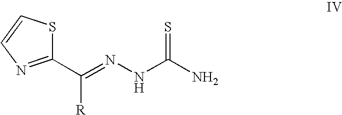
where R is H or C₁-C₄-alkyl.

Yet another preferred embodiment is a compound of formula V: 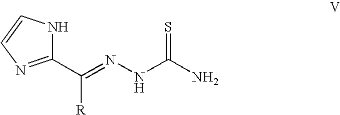
where R is R is H or C₁-C₄-alkyl

Finally, another preferred embodiment is a compound of Formula VI: 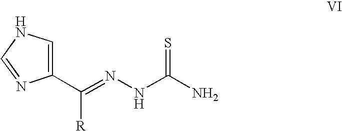
where R is H or C₁-C₄-alkyl.

As more preferred embodiments, the compounds of the present invention are selected from: 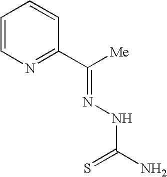
(of Formula II, where R is methyl, and R₁, R₂ and R₃ are H.)

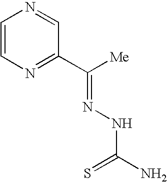

(of Formula III, where R is methyl and R₁ and R₂ are H.)

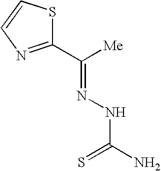

(of Formula IV, where R is methyl)

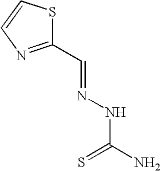

(of Formula IV, where R is H)

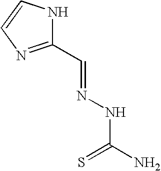

(of Formula V, where R is H) and

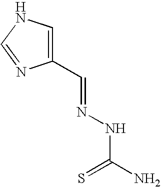

(of Formula VI, where R is H).

A most preferred embodiment of the present invention relates to methods of treating ischemia-related conditions by administering to a patient in need of such treatment PAN 811 (3-aminopyridine-2-carboxaldehyde thiosemicarbazone) of the following formula:

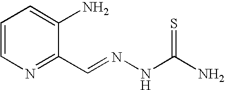

Certain of the compounds of the present invention may exist as E, Z-stereoisomers about the C═N double bond and the invention includes the mixture of isomers as well as the individual isomers that may be separated according to methods that are well known to those of ordinary skill in the art. Certain of the compounds of the present invention may exist as optical isomers and the invention includes both the racemic mixtures of such optical isomers as well as the individual entantiomers that may be separated according to methods that are well known to those of ordinary skill in the art.

Examples of pharmaceutically acceptable salts are inorganic and organic acid addition salts such as hydrochloride, hydrobromide, phosphate, sulphate, citrate, lactate, tartrate, maleate, fumarate, acetic acid, dichloroacetic acid and oxalate.

Examples of prodrugs include, for example, esters of the compounds with R₁-R₃ as hydroxyalkyl, and these may be prepared in accordance with known techniques.

It is surprising and unexpected that the inventors discovered that the compound, 3-aminopyridine-2-carboxaldehyde thiosemicarbazone, and several new analogs thereof, are effective as neuroprotectants, given that its only disclosed use thus far has been as an antineoplastic agent. See, for example, U.S. Pat. No. 5,721,259.

Thus, one of the embodiments of the present invention is directed to the amelioration of specific ischemia-related conditions, including but not limited to treatment of neuronal damage following global and focal ischemia from any cause (and prevention of further ischemic damage), treatment or prevention of otoneurotoxicity and of eye diseases involving ischemic conditions (such as, for example, macular degeneration), prevention of ischemia due to trauma or coronary bypass surgery, treatment or prevention of neurodegenerative conditions such as amyotrophic lateral sclerosis (ALS), Alzheimer's disease, Parkinson's disease, and Huntington's chorea, and treatment or prevention of diabetic neuropathies.

Reducing neuronal damage in the first minutes after a stroke is an important strategy to gain effective therapy. During stroke, the transport of oxygen and glucose to localized regions of the brain is halted by thromboembolic blockage of an artery, which causes neuronal loss in the central core of an infarction. The cells in the central core die very quickly via a necrotic mechanism. The area of the brain surrounding an ischemic infarct retains its structure, but is functionally (electrically) silent (known as “the penumbra”). The penumbra is a temporal zone, in that its evolution toward infarction is a relatively progressive phenomenon (Touzani et al., Curr. Opin. Neurol. 14:83-8, 2001). This zone provides the possibility of salvaging some of the brain function and the therapeutic window for treatment of the penumbra is much longer than that for the infarcted area.

The penumbra can also be described as a region of constrained blood supply in which energy metabolism is preserved. Therefore, the penumbra is a target of neuroprotective therapy, as well as for agents such as hyperbaric oxygen that would reactivate the dormant neurons. As such, immediate damage from injury in CNS trauma may not be reversible but the progression of a chain of events that would aggravate brain damage, predominantly global cerebral hypoxia/ischemia, can be prevented by an effective strategy for neuroprotection. For example, administration of a neuroprotectant before and/or during coronary artery bypass graft surgery (CABG, or bypass surgery) can effectively prevent neurodegeneration caused by the short-term decreases in blood flow to the brain (leading to a mild hypoxic/hypoglycemic state). The compounds of the present invention are capable of both significant neuroprotection as well as rescue of neurons after they have received damage, and thus are particularly useful in the administration of stroke victims.

The means for synthesis of compounds useful in the methods of the invention are well known in the art. Such synthetic schemes are described in U.S. Pat. Nos. 5,281,715; 5,767,134; 4,447,427; 5,869,676; and 5,721,259, all of which are incorporated herein by reference in their entireties.

In another aspect, the invention is directed to pharmaceutical compositions of the 2-caboxyaldehyde thiosemicarbazones useful in the methods of the invention. The pharmaceutical compositions of the invention comprise one or more of the compounds and a pharmaceutically acceptable carrier or diluent. As used herein “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

The pharmaceutical compositions of the present invention may be administered by any means to achieve their intended purpose, for example, by parenteral, subcutaneous, intravenous, intramuscular, intraperitoneal, transdermal, or buccal routes. Preferably, administration is oral, and may be of an immediate or delayed release. The dosage administered will be dependent upon the age, health, and weight of the recipient, kind of concurrent treatment, if any, frequency of treatment, and the nature of the effect desired, and such are typically determined by the clinician.

The pharmaceutical compositions of the present invention are manufactured by techniques common in the pharmaceutical industry, and the present invention is not limited hereby. The active agent(s) is/are preferably formulated into a tablet or capsule for oral administration, prepared using methods known in the art, for instance wet granulation and direct compression methods. The oral tablets are prepared using any suitable process known to the art. See, for example, Remington's Pharmaceutical Sciences, 18^{th} Edition, A. Gennaro, Ed., Mack Pub. Co. (Easton, Pa. 1990), Chapters 88-91, the entirety of which is hereby incorporated by reference. Typically, the active ingredient, one or more of the thiosemicarbazones, is mixed with pharmaceutically acceptable excipients (e.g., the binders, lubricants, etc.) and compressed into tablets. Preferably, the dosage form is prepared by a wet granulation technique or a direct compression method to form uniform granulates. Alternatively, the active ingredient(s) can be mixed with a previously prepared non-active granulate. The moist granulated mass is then dried and sized using a suitable screening device to provide a powder, which can then be filled into capsules or compressed into matrix tablets or caplets, as desired.

In one aspect, the tablets are prepared using a direct compression method. The direct compression method offers a number of potential advantages over a wet granulation method, particularly with respect to the relative ease of manufacture. In the direct compression method, at least one pharmaceutically active agent and the excipients or other ingredients are sieved through a stainless steel screen, such as a 40 mesh steel screen. The sieved materials are then charged to a suitable blender and blended for an appropriate time. The blend is then compressed into tablets on a rotary press using appropriate tooling.

Alternatively, the pharmaceutical composition is contained in a capsule containing beadlets or pellets. Methods for making such pellets are known in the art (see, Remington's, supra). The pellets are filled into capsules, for instance gelatin capsules, by conventional techniques.

Sterile injectable solutions can be prepared by incorporating a desired amount of the active compound in a pharmaceutically acceptable liquid vehicle and filter sterilized. Generally, dispersions are prepared by incorporating the active compound into a sterile vehicle containing a basic dispersion medium. In the case of sterile powders for the preparation of sterile injectable solutions, the preferred methods of preparation are vacuum drying and freeze-drying, which will yield a powder of the active ingredient plus any additional desired ingredient from a previously sterile-filtered solution thereof.

The active agent(s) in the pharmaceutical composition (i.e., one or more of the thiosemicarbazones) is present in a therapeutically effective amount. By a “therapeutically effective amount” is meant an amount effective, at dosages and for periods of time necessary, to achieve the desired therapeutic result of positively influencing the course of a particular disease state. Of course, therapeutically effective amounts of the active agent(s) may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects.

In another embodiment, the active agent is formulated in the composition in a prophylactically effective amount. By a “prophylactically effective amount” is meant an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

The amount of active compound in the composition may vary according to factors such as the disease state, age, sex, and weight of the individual. Dosage regimens may be adjusted to provide the optimum therapeutic response. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals. It is contemplated that the dosage units of the present invention will contain the active agent(s) in amounts about the same as those presently employed in antineoplastic treatment (e.g., Triapine®, Vion Pharmaceuticals, Inc.).

The pharmaceutical compositions of the invention may be administered to any animal in need of the beneficial effects of the compounds of the invention. Preferable the animal is a mammal, and most preferably, human.

This invention is further illustrated by the following examples, which are not intended to limit the present invention. The contents of all references, patents, and published patent applications cited throughout this application are specifically and entirely incorporated herein by reference.

### EXAMPLES

### Example 1

Comparison of the Neuroprotective Potencv of PAN-811 with Other Known Neuroprotectants

The purpose of this study was to compare the neuroprotective capacity of PAN-811 (3-aminopyridine-2-carboxaldehyde thiosemicarbazone; C₇H₉N₅S; MW=195) with known neuroprotectants, such as vitamin E, lipoic acid and Ginkgo biloba, in a cell-based model of Alzheimer's disease-associated oxidative stress.

Isolation and Acculturation of Cells.

Primary cortical neurons were isolated from a 17-day old rat embryonic brain and seeded on 96-well plate at 50,000 cells/well in regular neurobasal medium for 2-3 weeks. Twice, half the amount of medium was replaced with fresh neurobasal medium containing no antioxidants.

Treatment with PAN-811, Other Known Neuroprotectants and H₂O₂

PAN-811 was dissolved in EtOH at 1 mg/ml (˜5 mM), and further diluted in medium to final concentration at 0.1 μM, 1 μM, and 10 μM. The other known neuroprotectants were dissolved in appropriate solvents and diluted to the final concentrations as indicated. Neurons were pre-treated with PAN-811, known neuroprotectants, or control vehicle for 24 hours, and then subjected to oxidative stress induced by hydrogen peroxide (final concentration 150 μM). Controls included untreated cells (no compounds and hydrogen peroxide treatment), cells treated with compound only, and cells exposed to hydrogen peroxide but not compounds. Untreated cells were used as a control to evaluate both toxicity and viability of neurons. Each assay was performed in triplicate.

Evaluation of Cellular Function

After 24 hours, the cultures were evaluated for viability and mitochondrial function using a standard MTS Assay (Promega). The manufacturer's protocols were followed.

Materials

Neurobasal medium (Invitrogen); B27-AO, (Invitrogen); PAN-811 (Vion Pharmaceuticals); hydrogen peroxide (Calbiochem); EtOH (Sigma); Vitamin E (Sigma); lipoic acid (Sigma); Ginkgo biloba (CVS); MTS assay kit (Promega)

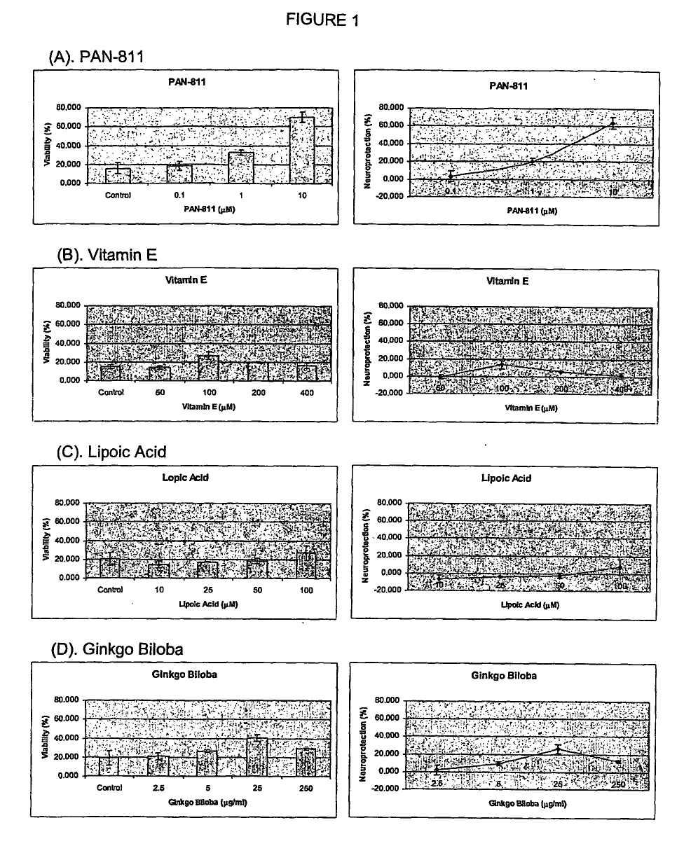

Experiments were carried out in accordance with the above study design. PAN-811 was dissolved in EtOH at 1 mg/ml (˜5 mM), and further diluted in neurobasal medium to final concentrations of 0.1 μM, 1 μM, and 10 μM. Lipoic acid was dissolved in EtOH at concentration 240 mM, and further diluted in the neurobasal medium to final concentrations of 10 μM, 25 μM, 50 μM and 100 μM. Vitamin E was dissolved in EtOH at a concentration of 100 mM, and further diluted in the neurobasal medium to final concentrations of 50 μM, 100 μM, 200 μM and 400 μM. Ginkgo biloba was dissolved in dH₂O at a concentration of 6 mg/ml, and further diluted in the neurobasal medium to final concentrations of 2.5 μg/ml, 5 μg/ml, 25 μg/ml, and 250 μg/ml. At the end of the treatment phase, the medium was replaced with 100-μl fresh, pre-warmed neurobasal medium plus B27 (-AO). The plates were returned to the incubator at 37° C. with 5% CO₂ for one hour. Subsequently, 20 μl MTS reagent was added to each well and the plates were incubated at 37° C. with 5% CO₂ for an additional two hours. The absorbance at 490 nm for each well was recorded with the BioRad plate reader (Model 550). Wells containing medium alone were used as blanks. Each data point is the average of three separate assay wells. Untreated cells were used as a control to calculate the cell viability and neuroprotective capacity. Two-week-old primary cultures were used for this set of study. See FIG. 1 for results.

Results

PAN-811 displayed good neuroprotective capacity at concentrations from 1-10 μM, even under harsh H₂O₂ treatment. Vitamin E and lipoic acid displayed minimal neuroprotective capacity under harsh treatment. Ginkgo biloba displayed a certain level of neuroprotection under harsh treatment.

PAN-811 displayed significant neuroprotection at 1-10 μM final concentration, even under harsh H₂O₂ treatment. The neuroprotective efficacy of PAN-811 significantly exceeded that of the other known neuroprotectants, Vitamin E, lipoic acid, and Ginkgo biloba.

### Example 2

Effect of PAN-811 on Reactive Oxygen Species (ROS) Generation in Neuronal Cells

The purpose of this study was to assess the capability of PAN-811 to reduce ROS generation in a cell-based model of Alzheimer's disease-associated oxidative stress.

Materials used in this example are the same as in Example 1.

Primary cortical neurons were isolated from a 17-day-old rat embryonic brain and seeded in 96-well plates at 50,000 cells/well in regular neurobasal medium for 2-3 weeks. Twice, half the amount of medium was replaced with fresh neurobasal medium without antioxidants.

The primary cortical neurons were rinsed once with HBSS buffer and incubated with 10 μM 5-(and-6)-chloromethyl-2′,7′-dichlorodihydrofluorescein diacetate, acetyl ester (CM-H₂DCFDA) to pre-load the dye. The cells were then rinsed with HBSS buffer once and treated with PAN-811 at final concentrations of 0.1, 1, 5, and 10 μM for 1 hour, and further subjected to oxidative stress induced by hydrogen peroxide at 300 μM for 2 hours.

c-DCF fluorescence at 485/520 nm (Ex/Em) for each well was recorded with a BMG Polar Star plate reader and used to evaluate ROS generation in cells. Untreated cells loaded with the dye were used as controls to calculate the c-DCF fluorescence change. Each assay was performed in triplicate.

Results

The c-DCF fluorescence at 485/520 nm (Ex/Em) for each well was recorded with the BMG Polar Star plate reader. Wells containing cells without dye were used as blanks. Each data point is the average of three separate assay wells. Untreated cells loaded with the dye were used as a control to calculate the c-DCF fluorescence change. Two-week-old primary cultures were used for the study.

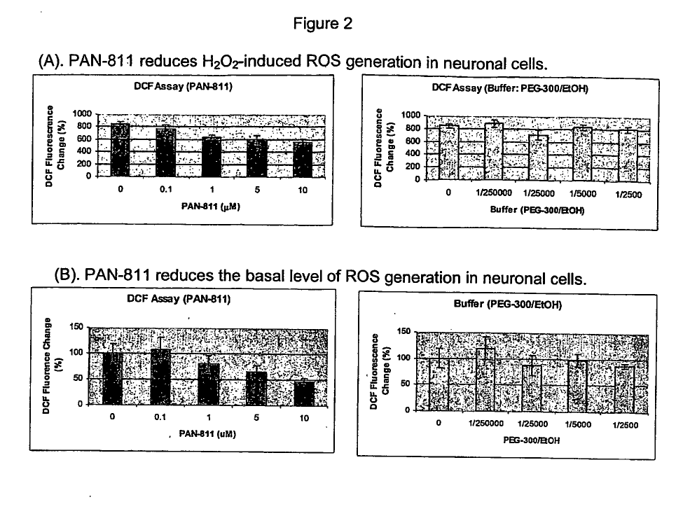

CM-H₂DCFDA is a cell-permeant indicator for reactive oxygen species (ROS), which is non-fluorescent until the acetate groups are removed by intracellular esterases and oxidation occurs within the cell. It has been widely employed to detect the generation of ROS in cells and animals. Here, it has been used as a tool to assess the effects of PAN-811 on ROS generation in neuronal cells following the procedures described in this example. As FIG. 2 illustrates, PAN-811 displayed good capacity to reduce H₂O₂-induced ROS generation, as well as basal level ROS generation in neuronal cells. The parallel control experiment using buffer, PGE-300/EtOH, instead of PAN-811, showed no effect on ROS generation in cells. Experiments were repeated four times in different batches of cells and similar results were obtained. See FIG. 2 for the representative experiment.

PAN-811 significantly reduced both H₂O₂-induced ROS generation (˜30% at 10 μM) and the basal level of ROS generation (˜50% at 10 μM) in primary neuronal cells.

Literature of Note:

Gibson G E, Zhang H, Xu H, Park L C, Jeitner T M. (2001). Oxidative stress increases internal calcium stores and reduces a key mitochondrial enzyme. Biochim Biophys Acta. March 16; 1586(2):177-89.

Chignell C F, Sik R H. (2003). A photochemical study of cells loaded with 2′,7′-dichlorofluorescin: implications for the detection of reactive oxygen species generated during UVA irradiation. Free Radic Biol Med. April 15; 34(8):1029-34.

### Example 3

PAN-811 is Neuroprotectant for Hypoxia- or Hypoxia/Hypoglycemia-Induced Neurotoxicity

The purpose of this example was to understand whether PAN-811 is able to protect hypoxia- or hypoxia/hypoglycemia (H/H)-induced neurotoxicity by examining its effects in vitro. As shown in the above examples, PAN-811 has been shown in related work to apply significant neuroprotection to primary neurons treated with H₂O₂.

The materials used in this example are the same as in Example 1. The LDH assay kit was obtained from Promega.

(Abbreviations: BSS=balanced salt solution; CABG=coronary artery bypass graft; d.i.v.=days in vitro; EtOH=ethanol; H/H=hypoxia/hypoglycemia; LDH=lactate dehydrogenase; MCAO=middle cerebral artery occlusion; NB=neurobasal medium; NMDA=N-methyl-D-aspartate; PEG=polyethylene glycol)

Experiments were performed in a 96-well plate format. Cortical neurons were seeded at a density of 50,000 cells/well on a poly-D-lysine coated surface, and cultured in serum-free medium (NB plus B27 supplement) to obtain cultures highly enriched for neurons. Neurons were cultured for over 14 d.i.v. to increase cell susceptibility to excitatory amino acids (Jiang et al., 2001). Six replicate wells were treated as a group to facilitate assay quantitation.

As shown in Table 1 below, glucose concentration normally is over 2.2 mM in the brain. It decreases to 0.2 mM and 1.4 mM in the central core and penumbra, respectively, during ischemia. Glucose levels return to normal 1 or 2 hours after recirculation (Folbergrová et al., 1995).

TABLE 1
Glucose Concentrations (mmol/kg)

| | | | 1-hour |
|---|---|---|---|
| | Sham | 2-hour MCAO | recirculation |
| Focus | 2.12 ± 0.18 | 0.21 ± 0.09 | 2.65 ± 0.19 |
| Penumbra | 2.20 ± 0.16 | 1.42 ± 0.34 | 2.69 ± 0.17 |

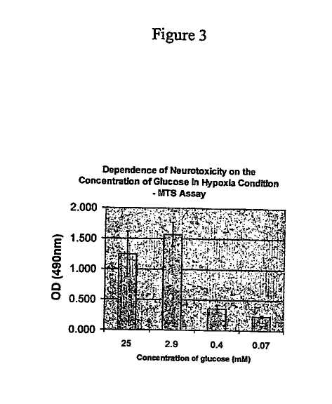

To understand the effect of glucose concentration on hypoxia-induced neurotoxicity, we tested different doses of glucose. As shown in FIG. 3, reduction of the glucose concentration to 2.9 mM did not result in neuronal cell death, by comparison to normal conditions where the glucose concentration is 25 mM. When glucose concentration went down to 0.4 mM, robust cell death occurred as indicated by the MTS assay.

To mimic the cerebral environments of a stroke, we established 3 in vitro model systems. The extreme H/H model (0.4 mM glucose) is a mimic of the environment in the central core of an infarct; the mild H/H model (1.63 mM glucose) is a mimic of the environment in the penumbra during MCAO; and the hypoxia only model (neurons in normal in vitro glucose concentration—25 mM) is a mimic of the environment in the penumbra after reperfusion since the possible cell death after reperfusion is predominantly a result of the hypoxic effect rather then energy failure.

Hypoxia/hypoglycemia was obtained by reducing glucose concentration down to 0.4 mM and 1.63 mM for extreme H/H and mild H/H, respectively. BSS (116.0 mM NaCl, 5.4 mM KCl, 0.8 mM MgSO4.7H₂O, 1.0 mM NaH2PO4, 1.8 mM CaCl2.2H₂O, 26.2 mM NaHCO3, and 0.01 mM glycine) or BSS with 25 mM glucose were de-gassed for 5 minutes prior to use. Culture medium in the plates for hypoxia was replaced with BSS or BSS with glucose. Meanwhile, culture medium in the plates for normoxia was replaced with non de-gassed BSS or BSS with glucose. Cells were committed to hypoxic conditions by transferring the plates into a sealed container (Modular Incubator Chamber-101™, Billups-Rothenberg, Inc.), applying a vacuum for 20 minutes to remove oxygen or other gases from the culture medium, and then refilling the chamber with 5% CO₂ and 95% N₂ at a pressure of 30 psi for 1 minute. The level of O₂ in the chamber was determined to be zero with an O₂ indicator (FYRITE Gas Analyzer, Bacharach, Inc.). Culture plates were maintained in the chamber for 6 hours. As an experimental control, duplicate culture plates were maintained under normal culture condition (5% CO₂ and 95% ambient air) for the same duration. After a 6-hour treatment, plates were removed from the chamber and the medium in both the hypoxic and normoxic cultures was replaced with a termination solution (DMEM supplemented with 1× sodium pyruvate, 10.0 mM HEPES, and 1×N₂ supplement) containing 25 mM glucose and cultured in 5% CO₂ and 95% ambient air conditions. Neurons were treated with varying concentrations of PAN-811 or vehicle as a negative control. MK801 was utilized as a positive control. Mitochondrial function and cell death were evaluated at 24 or 48 hours post H/H insult with the MTS and LDH analyses (see below).

In the sole hypoxia model, the neurons were pre-treated with solvent or PAN-811 for 24 or 48 hours. Treatment with drug was continued during and subsequent to a 24-hour period of hypoxia. Cellular morphology and function (MTS and LDH assays) were measured 24 or 48 hours subsequent to the hypoxic insult.

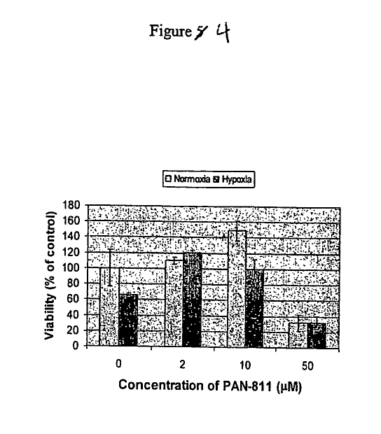

Neuronal cell death evaluated morphologically as seen in FIG. 4. Neurons prior to hypoxia are healthy with phase-brilliant cell soma (arrow head) and intact neuronal processes (open arrow). The processes and their branches form a dense network in the background. Hypoxia causes shrinkage of the cell body and collapse of the neuronal processes and network. PAN-811, as well as the glutamate NMDA receptor antagonist MK801 at doses of 5 μM, shows efficient protection from neuronal cell death and partial reservation of the neuronal processes.

The MTS assay is a calorimetric assay that measures the mitochondrial function in metabolically active cells. This measurement indirectly reflects cell viability. The MTS tetrazolium compound is reduced in metabolically active mitochondria into a colored formazan product that is soluble in tissue culture medium, and can be detected via its absorbance 490 nm. 20 μl of MTS reagent (Promega) are added to each well of the 96 well assay plates containing the samples in 100 μl of culture medium. The plate is then incubated in a humidified, 5% CO₂ atmosphere at 37° C. for 1-2 hours until the color is fully developed. The absorbance at 490 nm was recorded using a Bio-Rad 96 well plate reader.

The lactate dehydrogenase (LDH) assay is based on the reduction of NAD by the action of LDH. The resulting reduced NAD (NADH) is utilized in the stoichiometric conversion of a tetrazolium dye. If cell-free aliquots of medium from cultures given different treatments are assayed, then the amount of LDH activity can be used as an indicator of relative cell death as well as a function of membrane integrity. A 50 μl aliquot of culture medium from a well in tested 96-well plate is transferred into a well in unused plate and supplemented with 25 μl of equally-mixed Substrate, Enzyme and Dye Solutions (Sigma). The preparation is incubated at room temperature for 20-30 minutes, and then measured spectrophotometrically at wavelength of 490 nm.

Results

Sole Hypoxia Model

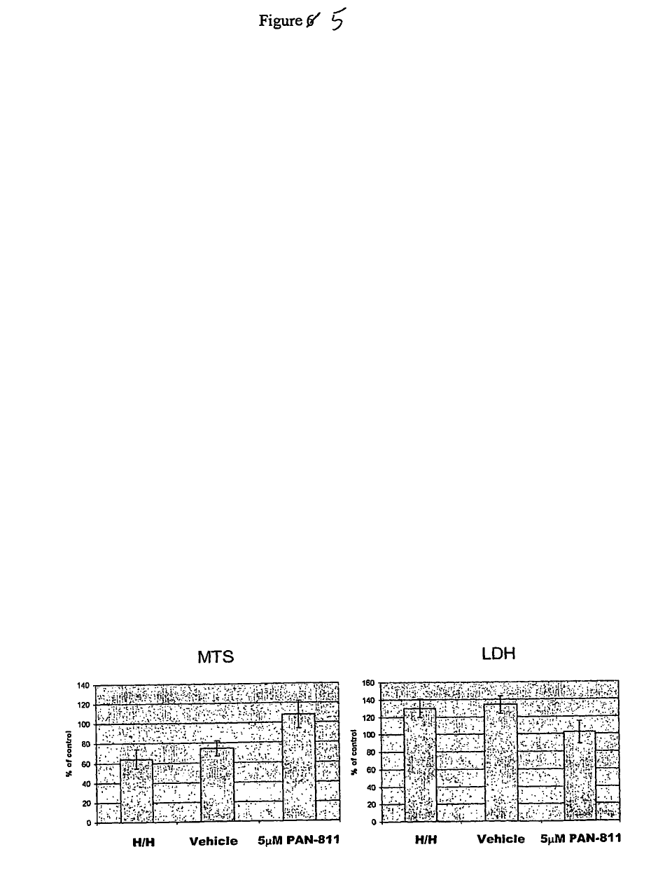

Cortical neurons were treated with PAN-811 for 48-hour prior to hypoxia; PAN-811 remained present during 24-hour hypoxia and for a 48-hour period subsequent to hypoxia. PAN-811 at dose of 2 μM completely blocked the cell death but 50 μM was toxic (see FIG. 5).

Cortical neurons were treated with 2 μM PAN-811, 1:80 green tea or 5 μM MK801 for 24 hours prior to, during and subsequent to a 24-hour period of hypoxia. PAN-811 demonstrated highest efficacy among reagents tested, completely blocking neuronal cell death and mitochondrial dysfunction.

Mild H/H Model

PAN-811 protected neurons from mild H/H-induced neurotoxicity before and during insult.

Embryonic (E17) rat cortical neurons were cultured for 15 days, treated with PAN-811 and vehicle 24-hours before and during hypoxia/hypoglycemia (6-hours). MTS and LDH assays were carried out 17 hours post to the insults. PAN-811 at 5 μM, but not a 1:1,520 dilution of PEG:EtOH (which corresponds to the mount of vehicle in 5 μM PAN-811), completely protected hypoxia/hypoglycemia-induced mitochondria dysfunction and neuronal cell death.

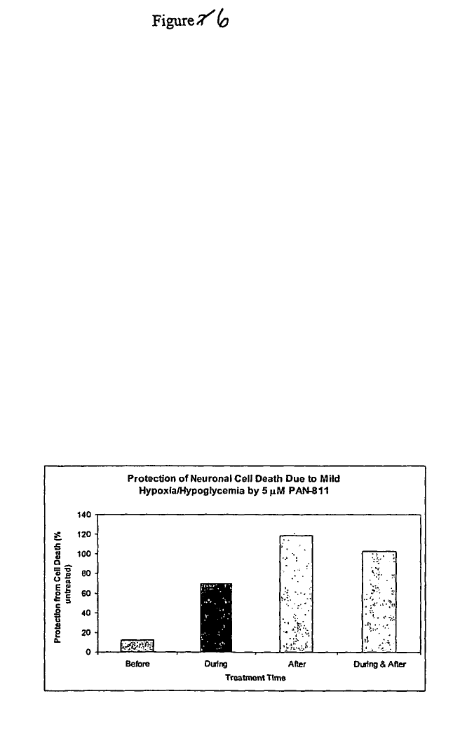

The data shown in FIG. 6 are representative. A summary of 6 experiments that cover a concentration range of 2-50 μM is shown in the following Table 2.

TABLE 2

| | Culture age | Pre- | | |
|---|---|---|---|---|
| | Comments | treatment | H/H duration | Post to H/H |
| Date | (days) | (hours) | (hours) | (hours) |
| Apr. 17, 2003 | 13 | 24 | 6 | 48 |
| | 2 μM: | | | |
| | 100% protected | | | |
| May 2, 2003 | 22 | 24 | 6 | 24 |
| | 2 μM: | | | |
| | 100% protected | | | |
| May 8, 2003 | 42 | 24 | 6 | 24 |
| | 2 μM: | | | |
| | 100% protected | | | |
| Jul. 9, 2003 | 13 | 24 | 6 | 20 |
| | 2 μM: | | | |
| | 100% protected | | | |
| Jul. 13, 2003 | 15 | 24 | 6 | 24 |
| | 10 μM: | | | |
| | 100% protected | | | |
| Jul. 25, 2003 | 15 | 24 | 6 | 24 |
| | 5 μM: | | | |
| | 100% protected | | | |

** Test range started from 5 μM for the experiments of Jul. 13, 2003 and Jul. 25, 2003

PAN-811 protected cells from mild H/H-induced neurotoxicity during and especially after the insults.

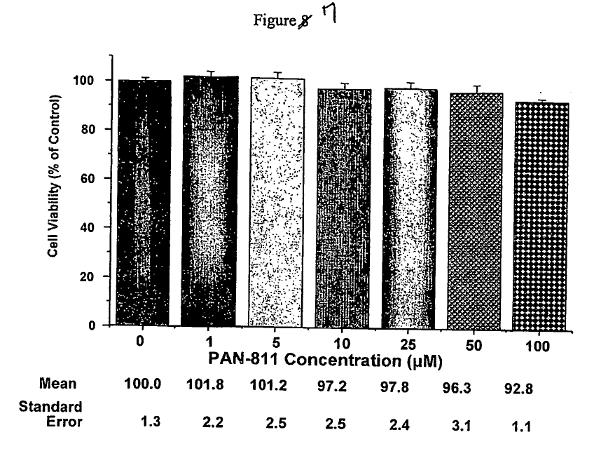

The neurons were cultured for 15 days, and treated with PAN-811 or PEG:EtOH (7:3) as vehicle for a 24-hour period prior to 6-hour H/H (Before Group). Alternatively the neurons were cultured for 16 days, and then treated with above reagents during 6-hour H/H (During Group), treated for a 6-hour H/H period and 48-hour period subsequent to the H/H (During and After Group), or treated for a 48-hour period subsequent to the H/H (After group). The LDH assay was carried out 48 hours after the period of H/H. The results demonstrated that PAN-811 protected neuronal cell death when treating the neurons during and especially after H/H, but marginally before H/H, see FIG. 7.

Extreme H/H Model

PAN-811 at ≦50 μM did not protect neuronal cell death (data not shown).

PAN-811 at 2 μM completely protected sole hypoxia- and mild H/H induced neurotoxicity. PAN-811 at 100 μM only partially blocked extreme H/H-induced neuronal cell death so PAN-811 is unlikely to be involved in energy metabolism.

PAN-811 significantly protects neurons from cell death when administered either during or subsequent to a hypoxic or ischemic insult.

The efficacy of PAN-811 is significantly greater than that of MK801 and/or green tea.

PAN-811 at 50 μM is toxic to neurons in long-term exposure (120-hour exposure).

Literature of Note:

Jiang, Z.-G., Piggee, C. A., Heyes, M. P., Murphy, C. M., Quearry, B., Zheng, J., Gendelman, H. E., and Markey, S. P. Glutamate is a principal mediator of HIV-1-infected immune competent human macrophage neurotoxicity. J. Neuroimmunology 117(1 2):97-107, 2001.

Folbergrová, J., Zhao, Q., Katsura, K., and Siesjö, B. K. N-tert-butyl-phenylnitrone improves recovery of brain energy state in rats following transient focal ischemia. Proc. Natl. Acad. Sci. USA 92:5057-5061, 1995.

### Example 4

PAN-811 Displays Significant Neuroprotection in an In Vivo Model of Transient Focal Brain Ischemia

PAN-811 has shown significant neuroprotection in in vitro models of oxidative stress and ischemia. This work, coupled with the known toxicity profile and pharmacokinetic data on the compound, are highly compatible with its use in the treatment of stroke.

Materials are the same as those used in the above examples. In this example, MCAO is used as the abbreviation for middle cerebral artery occlusion.

Prior to embarking on in vivo studies, PAN-811 was tested in several cellular models of neurodegeneration.

Enriched neuronal cultures were prepared from 15-day-old Sprague-Dawley rat embryos. Using aseptic techniques, the rat embryos were removed from the uterus and placed in sterile neuronal culture medium. Using a dissecting microscope, the brain tissue was removed from each embryo, with care taken to discard the meninges and blood vessels. The cerebellum was separated by gross dissection under the microscope, and only cerebellar tissue was used for the culture. Cells were dissociated by trituration of the tissue and were plated at a density of 5×10⁵ cells/well onto 48-well culture plates precoated with poly(L-lysine). Cultures were maintained in a medium containing equal parts of Eagle's basal medium (without glutamine) and Ham's F-12k medium supplemented with 10% heat-inactivated horse serum, 10% fetal bovine serum, 600 μg/ml glucose, 100 μg/ml glutamine, 50 U/ml penicillin, and 50 μg/ml streptomycin. After 48 h, 10 μM cytosine arabinoside was added to inhibit non-neuronal cell division. Cells were used in experiments after 7 days in culture.

Cells were treated with varying amounts of PAN-811 (0-100 μM) for 24 hrs. Cell viability was determined in the MTT assay.

Four in vitro models of excitotoxicity were studied. Cells were either exposed to H/H conditions for 3 hrs or treated for 45 min with one of glutamate (100 μM), staurosporine (1 μM) or veratridine (10 μM). All cells were co-treated with or without PAN-811 (10 μM) in Locke's solution. At the conclusion of the respective excitotoxic exposures, the condition medium (original) was replaced. H/H was induced by incubating the cells in a humidified airtight chamber saturated with 95% nitrogen, 5% CO2 gas for 3 hrs in Locke's solution without added glucose.

Twenty-four hours after the excitotoxic insult, cell viability assessments were made. Cell damage was quantitatively assessed using a tetrazolium salt colorimetric assay with 3-(4,5-dimethylthiazol-2-yl)-2,5-diphenyltetrazolium (MTT; Sigma Chemical Co., St. Louis, Mo.). Briefly, the dye was added to each well (final concentration, 1.5 mg/ml), cells were incubated with MTT-acidified isopropanol (0.1 N HCl in isopropanol), and the absorbance intensity (540 nm) of each sample was measured in a 96-well plate reader. Values are expressed relative to vehicle-treated control cells that were maintained on each plate, and the percentage change in cell viability was calculated.

In Vivo Studies.

Thirty-six male Sprague-Dawley rats (270-330 g; Charles River Labs, Raleigh, Va.) were used in this study. Anesthesia was induced by 5% halothane and maintained at 2% halothane delivered in oxygen. Body temperature was maintained normothermic (37±1° C.) throughout all surgical procedures by means of a homeothermic heating system (Harvard Apparatus, South Natick, Mass.). Food and water were provided ad libitum before and after surgery, and the animals were individually housed under a 12-h light/dark cycle. Rats were anesthetized and prepared for temporary focal ischemia using the filament method of middle cerebral artery occlusion (MCAO) and reperfusion. Briefly, the right external carotid artery was isolated and its branches were coagulated. A 3-0 uncoated monofilament nylon suture with a rounded tip was introduced into the internal carotid artery via the external carotid artery and advanced (approximately 22 mm from the carotid bifurcation) until a slight resistance was observed, thus occluding the origin of the MCA. The endovascular suture remained in place for 2 h and then was retracted to allow reperfusion of blood to the MCA. After MCAO surgery, animals were placed in recovery cages with ambient temperature maintained at 22° C. During the 2-h ischemia period and the initial 6-h post-ischemia period, 75-W warming lamps were also positioned directly over the top of each cage to maintain body temperature normothermic throughout the experiment.

The rats were treated 10 minutes prior to MCAO with 1/mg/kg PAN-811 via IV injection. PAN-811 was prepared as a stock solution in 70% PEG300, 30% EtOH. This stock was diluted 5-fold in sterile saline prior to injection (final concentration 1 mg/ml).

For each rat brain, analysis of ischemic cerebral damage was measured as a function of total infarct volume. This was achieved using 2,3,5-triphenyl tetrazolium chloride (TTC) staining from seven coronal sections (2-mm thick). Brain sections were taken from the region beginning 1 mm from the frontal pole and ending just rostral to the corticocerebellar junction. Computer-assisted image analysis was used to calculate infarct volumes. Briefly, the posterior surface of each TTC-stained forebrain section was digitally imaged (Loats Associates, Westminster, Md.) and quantified for areas (in square millimeters) of ischemic damage.

Results

In Vitro Studies

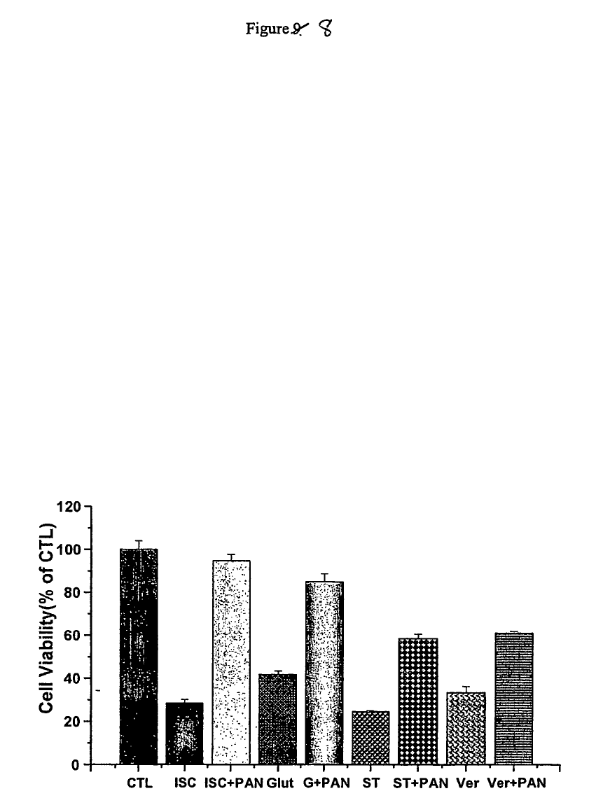

Neurotoxicity of PAN-811. Results are presented in FIG. 1. Essentially, PAN-811 showed only slight toxicity at concentrations up to 100 μM. Maximal toxicity was only 7.8% at the highest concentration tested (see FIG. 8).

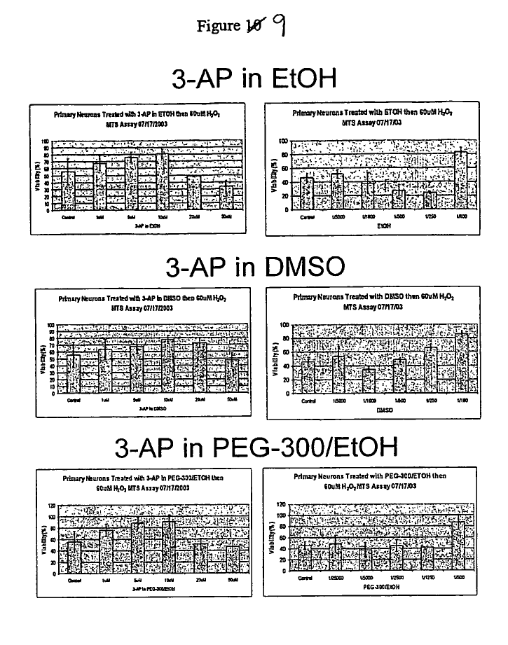

Neuroprotection due to PAN-811. PAN-811 was found to significantly protect neurons from for different excitotoxic insults (FIG. 2). Pre-treatment of neurons with 10 μM PAN-811 protected cells from the damage induced by a 3-hour period of hypoxia/hypoglycemia (92% protection), from 100 μM glutamate (˜75%), 1 μM staurosporine, an inhibitor of protein kinase C and inducer of apoptosis (˜47%) and 10 μM veratridine a sodium channel blocker (˜39%). See FIG. 9.

In Vivo Studies.

Results of this experiment are presented in Table 3. In total, 36 rats were used for the experiment, however 11 rats were excluded due to the following reasons: 4 rats died of severe stroke without complications of hemorrhage, 4 rats were excluded due to sub acute hemorrhage (3 of them died<24 h), 1 rat was excluded due to a fire drill during surgery, 1 rat was excluded due to being statistical outlier, and 1 rat died of overdose of halothane. Of the 7 rats that died (4 from severe strokes without SAH, and 3 with SAH), 6 were untreated (vehicle) rats and only 1 was treated with PAN-811. Vehicle treated rats had a mean infarct volume of 292.96 mm³ with a range from 198.75-355.81. PAN-811 treated rats had a mean infarct volume of 225.85 mm³ with a range 42.36-387.08. This represents a neuroprotection of 23% (p<0.05). For reasons yet to be determined, more severe injury was noted in the control group than is normally measured. Accordingly, the infarct size for the PAN-811 treated animals is also larger than expected for significant neuroprotection. Despite this issue the variability in both treatment groups was excellent (10% or less of the SEM) and was as good, if not better, than most of our previously published studies.

PAN-811 is well tolerated and relatively non-toxic in both the in vitro and in vivo model systems.

Pre-treated of neurons with 10 μM PAN-811 gave significant protection against for excitotoxic insults that result in neurodegeneration.

Pre-treatment of rats 10 minutes prior to a period of transient focal brain ischemia with a single dose of PAN-811 (1 mg/kg) yielded a 23% reduction in average infarct volume.

Literature of Note:

Williams A J, Dave J R, Phillips J B, Lin Y, McCabe R T, and Tortella F C. (2000) Neuroprotective efficacy and therapeutic window of the high-affinity N-methyl-D-aspartate antagonist conantokin-G: in vitro (primary cerebellar neurons) and in vivo (rat model of transient focal brain ischemia) studies. J Pharmacol Exp Ther. July; 294(1):378-86.

TABLE 3

| | |
|---|---|
| Vehicle Treated | PAN-811 |

| | Infarct | | Infarct |
|---|---|---|---|
| Rat # | Volume | Rat # | Volume |
| R28 | 198.75 | R21 | 42.36 |
| R17 | 208.03 | R1 | 126.42 |
| R2 | 267.38 | R30 | 143.74 |
| R11 | 270.89 | R24 | 158.83 |
| R34 | 282.51 | R3 | 196.18 |
| R19 | 308.19 | R26 | 200.08 |
| R27 | 308.45 | R23 | 218.54 |
| R36 | 334.81 | R20 | 221.46 |
| R10 | 339.85 | R25 | 224.32 |
| R4 | 347.89 | R31 | 255.36 |
| R32 | 355.81 | R5 | 267.40 |
| | | R13 | 344.47 |
| | | R16 | 375.59 |
| | | R8 | 387.08 |
| Mean | 292.96 | Mean | 225.85 |
| SD | 53.60 | SD | 96.67 |
| SEM | 16.16 | SEM | 25.84 |
| N | 11 | n | 14 |

| | |
|---|---|
| p value | 0.05 |
| % protection | 23% |

Table I: Infarct Volume in mm³ of vehicle and PAN-811 treated rats. Rats were treated with 1 mg/kg PAN-811 10 minutes prior to MCAO. Infarct volume was determined 24 hours after surgery.

### Example 5

Protection of Neurons from H₂O₇-Induced Oxidative Stress by PAN-811

The purpose of this study was to assess the efficacy of PAN-811 as a neuroprotectant in a cell-based model of Alzheimer's disease-associated oxidative stress. Neuroprotection and cellular toxicity are determined. Various solvents were tested to determine their appropriateness as vehicles for the delivery of PAN-811.

The materials are the same as in the other examples.

Primary cortical neurons were isolated from a 17-day-old rat embryonic brain and seeded on 96-well plate at 50,000 cells/well in regular neurobasal medium for 2-3 week. Twice, half amount of medium was replaced with fresh neurobasal medium containing no antioxidants.

PAN-811 was dissolved in either EtOH or DMSO at 1 mg/ml (˜5 mM), in PEG-300/EtOH (70%/30%) at 5 mg/ml (˜25 mM), and further diluted in medium to final concentration at 1 μM, 5 μM, 20 μM and 50 μM. Neurons were pre-treated with PAN-811 or vehicle for 24 hours, and then subjected to oxidative stress induced by hydrogen peroxide (final concentration 60-70 μM). Controls include untreated cells (no PAN-811 and hydrogen peroxide treatment), cells treated with PAN-811 only, and cells exposed to hydrogen peroxide but not PAN-811. Untreated cells were used as a control to evaluate both toxicity and improved viability of neurons. Each assay was performed in triplicate. Equal volume of solvents (EtOH, DMSO, and PEG-300/EtOH) was added to cells to test the solvent effects on the assay.

After 24 hours, the cultures were evaluated for viability and mitochondrial function using a standard MTS Assay (Promega). The manufacturer's protocols were followed.

Results

Experiment 1

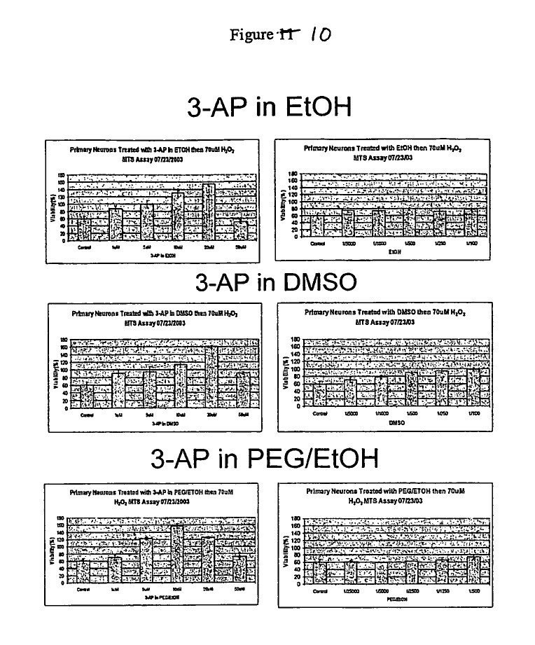

At the end of the treatment, all media were replaced with 100 μl fresh pre-warmed neurobasal medium plus B27 (-AO). The plates were put back into the incubator at 37° C. with 5% CO₂ for one hour, then 20 μl MTS reagent was added to each well and plates were incubated at 37° C. with 5% CO₂ for an additional two hours. The absorbance at 490 nm for each well was recorded with the BioRad plate reader (Model 550). Wells containing media alone were used as blanks. Each data point is the average of three separate assay wells. Untreated cells were used as a control to calculate the cell viability and neuroprotective capacity. Three-week-old primary cultures were used for this set of study. See FIG. 10 for results.

Experiment 2

Experiments were carried out following the same procedures as experiment 1. Two-week-old primary cultures were used for this study. See FIG. 11 for results.

In these experiments, all three solvents showed minimal effects on the assay system at dilutions corresponding to final PAN-811 concentrations from 1-10 μM. DMSO displayed a certain level of neuroprotection at dilutions corresponding to final PAN-811 concentrations at or above 20 μM. EtOH and PEG-300/EtOH showed a certain level neuroprotection capacity at the dilution corresponding to a 50 μM final concentration of PAN-811. PAN-811 showed good neuroprotective capacity at 1-10 μM. PAN-811 has better solubility in PEG-300/EtOH comparing to EtOH alone.

PAN-811 showed good neuroprotective capacity at 1-10 μM final concentration. PEG-300/EtOH showed very minimal interference with the assay system at dilutions corresponding to 1-20 μM of PAN-811, and is thus the best solvent for PAN-811 among the three solvents tested.

Those of skill in the art will readily appreciate that the present invention is well adapted to carry out the objects and obtain the ends and advantages mentioned, as well as those inherent therein, and would know that various modifications and variations can be made in practicing the present invention without departing from the spirit or scope of the invention. Such modifications and variations are considered by the inventors as encompassed within the spirit of the invention, which is further defined in the appended claims.

We claim:

1. A method of ameliorating, treating or preventing neuronal damage due to ischemic conditions, comprising administering to a subject in need thereof a therapeutically active amount of a compound of Formula I, or a pharmaceutically acceptable salt or prodrug thereof: 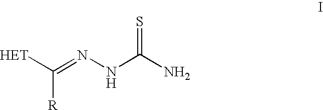
where HET is a 5 or 6 membered heteroaryl residue having 1 or 2 heteroatoms selected from N and S, and optionally substituted with an amino group; and R is H or C₁-C₄-alkyl.

2. The method of claim 1, wherein the compound, or a salt or prodrug thereof, administered to the subject is of Formula II: 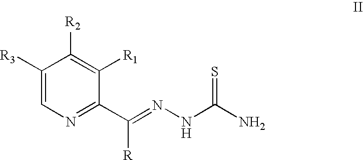
where R is H or C₁-C₄-alkyl; and R₁, R₂ and R₃ are independently selected from H and amino.

3. The method of claim 1, wherein the compound, or a salt or prodrug thereof, administered to the subject is of Formula III: 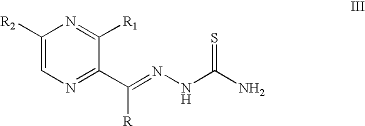
where R is H or C₁-C₄-alkyl; and R₁ and R₂ are independently selected from H and amino.

4. The method of claim 1, wherein the compound, or a salt or prodrug thereof, administered to the subject is of Formula IV: 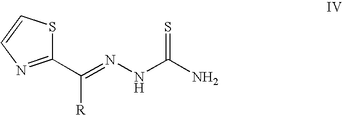
where R is H or C₁-C₄-alkyl.

5. The method of claim 1, wherein the compound, or a salt or prodrug thereof, administered to the subject is of Formula V: 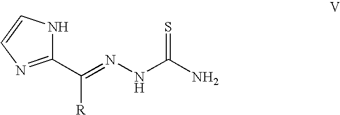
where R is H or C₁-C₄-alkyl.

6. The method of claim 1, wherein the compound, or a salt or prod rug thereof, administered to the subject is of Formula VI: 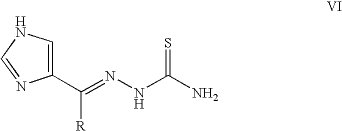
where R is H or C₁-C₄-alkyl.

7. The method of claim 1, wherein the compound, or a salt or prodrug thereof, administered to the subject is

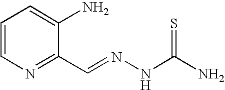

8. The method of claim 2, wherein R is methyl and R₁, R₂ and R₃ are H.

9. The method of claim 3, wherein R is methyl and R₁ and R₂ are H.

10. The method of claim 4, wherein R is methyl.

11. The method of claim 5, wherein R is H.

12. The method of claim 6, wherein R is H.

13. A compound of Formula I, wherein HET is a 5 or 6 membered unsubstituted heteroaryl residue having 1 or 2 heteroatoms selected from N and S; and R is H or C₁-C₄-alkyl.

14. The compound of claim 13, HET is a pyridine, pyrazine, thiazole or imidazole.

15. The compound of claim 14, wherein R is methyl.

14. A pharmaceutical composition comprising one or more of the compounds according to claims 13, 14 or 15, together with a pharmaceutically acceptable carrier.
